(19) United States
(12) Patent Application Publication
(10) Pub. No.: US 2019/0209083 A1
(43) Pub. Date: Jul. 11, 2019

(54) MONITORING SYSTEM AND MONITORING METHOD FOR INFANT

(71) Applicant: NATIONAL CHIAO TUNG UNIVERSITY, Hsinchu City (TW)

(72) Inventors: Bing-Fei Wu, Hsinchu City (TW); Kuan-Hung Chen, Hsinchu City (TW); Meng-Liang Chung, Changhua County (TW); Po-Wei Huang, Yunlin County (TW); Tsong-Yang Tsou, Taoyuan City (TW); Yun-Wei Chu, Taichung City (TW); Han-Kuang Kao, Taichung City (TW)

(21) Appl. No.: 16/243,042

(22) Filed: Jan. 8, 2019

(30) Foreign Application Priority Data
Jan. 9, 2018 (TW) 107100805

(51) Int. Cl. A61B 5/00; A61B 5/0205; A61B 5/1171

(52) U.S. Cl. CPC A61B 5/4818; A61B 5/0205; A61B 5/1176; A61B 5/02427; A61B 5/7282; A61B 5/746; A61B 5/0077; A61B 5/6889

(57) ABSTRACT

A monitoring system for infant includes image sensors, a face region determination module, a human face identification module, a non-contact physiological information measurement module and a physiological information determination module. The image sensors are configured to capture images consecutively. The face region determination module is configured to determine whether images include a face region. When not include, the face region determination module outputs a first warning information and when include, the human face identification module is configured to identify a monitored person in images to extract the historical information. The non-contact physiological information measurement module is configured to calculate color difference of each pixel of the face region, output heartbeat waveform and calculate physiological information. The physiological information determination module is configured to determine whether a heart rate, variation of heart rate and respiration information in the physiological information are in a reference range and outputs a second warning information.

### CROSS-REFERENCE TO RELATED APPLICATION

This application claims priority to Taiwan Application Serial Number 107100805, filed Jan. 9, 2018, which is herein incorporated by reference in its entirety.

### BACKGROUND

### Technical Field

The present disclosure relates to a monitoring system and a monitoring method. More particularly, the present disclosure relates to a monitoring system and a monitoring method for infant with warning function.

### Description of Related Art

In recent years, the country gradually entered a low fertility society. The health care for infants draws more attention. In view of the accidents of sudden infant death during sleeping occurring again and again, for parents or nursing staffs, it is difficult to take care of infants anywhere and all the time. As a result, using monitoring system to monitor infants becomes a trend in the future gradually.

Nevertheless, for monitoring physiological information or other physiological information, the monitoring system in the prior art is usually used by attaching sensors on infants' skin directly. Furthermore, in order to prevent the sensor from being removed by the infants' movement, the sensors are attached on the infants' skin by fastening approaches such as gel, tapes, etc., but there is high possibility of causing injury to the infants' skin.

### SUMMARY

One aspect of the present disclosure is to provide a monitoring system and a monitoring method for infant, utilizing a non-contact way to monitor the physiological information.

Another aspect of the present disclosure is to provide a monitoring system and a monitoring method for infant, capable to issue warning information.

In the present disclosure a monitoring system for infant includes one or more image sensors, a face region determination module, a human face identification module, a non-contact physiological information measurement module and a physiological information determination module. One or more image sensors are configured to capture a plurality of images consecutively. The face region determination module is configured to determine whether each of the images includes a face region, wherein when each of the images does not include the face region, the face region determination module outputs a first warning information. The human face identification module, when each of the images includes the face region, is configured to identify the identity of a monitored person in the images in order to extract the past historical information corresponding to the identity of the monitored person. The non-contact physiological information measurement module, when each of the images includes the face region, is configured to calculate a color difference of each pixel of the face region of the images captured in sequence to output a heartbeat waveform and calculate, according to the heartbeat waveform, a required physiological information. The physiological information determination module is configured to determine a heart rate, variation of the heart rate, respiration information included in the physiological information, wherein when the physiological information is not in a reference range, the physiological information determination module outputs a second warning information.

In the present disclosure a monitoring method for infant includes: capturing a plurality of images consecutively, determining whether each of the images includes a face region, when each of the images does not include the face region, outputting a first warning information, when each of the images includes the face region, identifying the identity of a monitored person in order to extract the past historical information, when each of the images includes the face region, calculating a color difference of each pixel of the face region of the images captured in sequence to output a heartbeat waveform, and calculating, by the heartbeat waveform, a required physiological information and determining whether a heart rate, variation of the heart rate and respiration information included in the physiological information are in a reference range, when the physiological information is not in the reference range, outputting a second warning information.

### BRIEF DESCRIPTION OF THE DRAWINGS

FIG. 1 is a function block diagram of a monitoring system for infant, according to one embodiments of the present disclosure.

FIG. 2 is a flowchart of a monitoring method for infant, according to one embodiments of the present disclosure.

FIG. 3 is a schematic diagram of the monitoring system for infant monitoring a monitored person, according to one embodiments of the present disclosure.

FIG. 4 is a schematic diagram of an image, according to one embodiments of the present disclosure.

FIG. 5 is a schematic diagram of a range of a face region, according to one embodiments of the present disclosure.

FIG. 6A to FIG. 6C are the schematic diagrams of a plurality of images, without the face region, captured by a sensor of the monitoring system for infant, according to one embodiments of the present disclosure.

FIG. 7A to FIG. 7C are the schematic diagrams of a plurality of images, the face region within, captured by the sensor of the monitoring system for infant, according to one embodiments of the present disclosure.

FIG. 8 is a further function block diagram of a non-contact physiological information measurement module of the monitoring system for infant, according to one embodiments of the present disclosure.

FIG. 9 is a further flowchart of a step S160 of the monitoring method for infant, according to one embodiments of the present disclosure.

FIG. 10A to FIG. 10C are the schematic diagrams of a region of interest according to one embodiments of the present disclosure.

FIG. 11 is a flowchart of a monitoring method for infant, according to another embodiment of the present disclosure.

### DETAILED DESCRIPTION

The following embodiments are disclosed with accompanying diagrams for detailed description and ease of understanding. However, it should be understood that these details of given embodiments do not intend to limit the present disclosure and the descriptions of construction operations do not limit the order of the execution. The equivalent constructions by reconfiguration of the components do not depart from the spirit and scope of the present disclosure.

It should be noted that the module illustrated in the present disclosure can be implemented with a circuit or a circuitry. The present disclosure is not limited thereto.

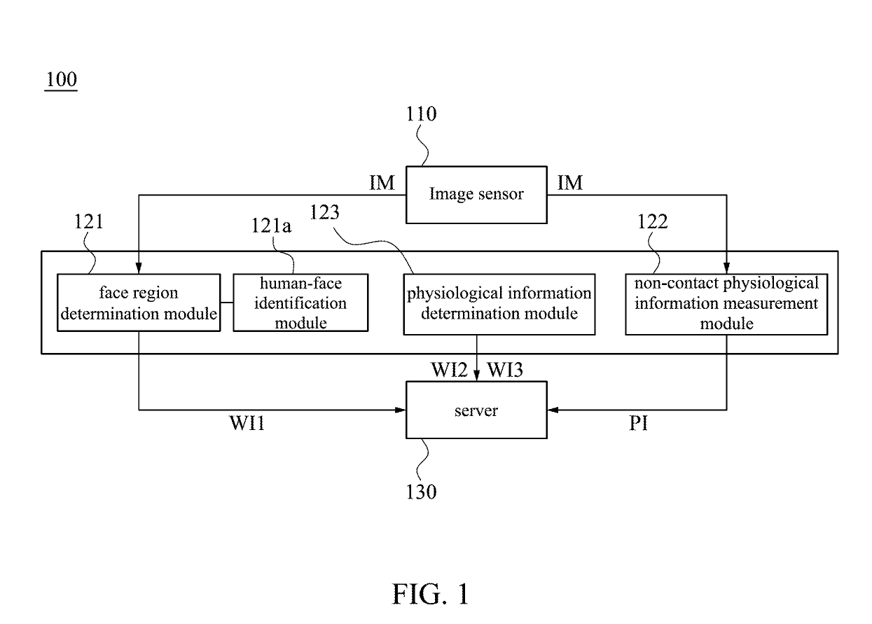

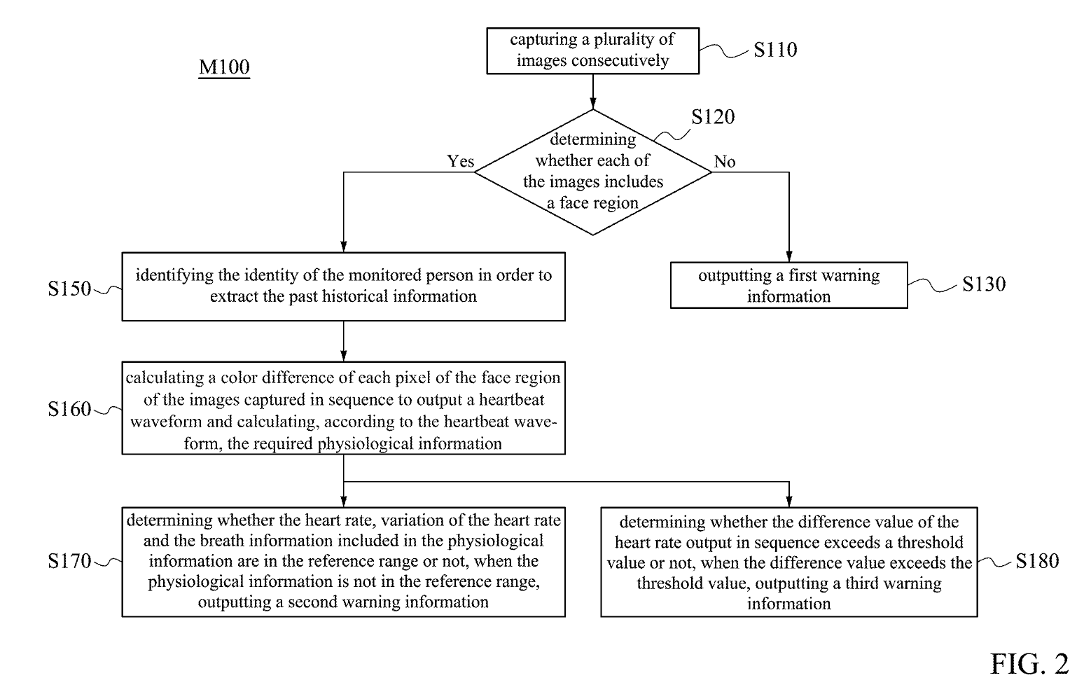

Reference is now made to FIG. 1 and FIG. 2. FIG. 1 is a function block diagram of a monitoring system 100 for infant, according to one embodiments of the present disclosure. FIG. 2 is a flowchart of a monitoring method M100 for infant, according to one embodiments of the present disclosure.

In present embodiment, the monitoring system 100 for infant is configured to apply the monitoring method M100 for infant to perform monitoring, wherein the monitoring system 100 for infant includes an image sensor 110, a face region determination module 121, a human face identification module 121a, a non-contact physiological information measurement module 122, a physiological information determination module 123 and a server 130. The monitoring method M100 for infant includes steps S110, S120, S130, S160, S170 and 180.

Furthermore, the monitoring system 100 for infant can be configured to monitor instantly a physiological condition of a monitored person lying down. Once the physiological condition of the monitored person is abnormal, the monitoring system 100 for infant sends a warning information to inform relevant people to handle the situation, so as to avoid accidents.

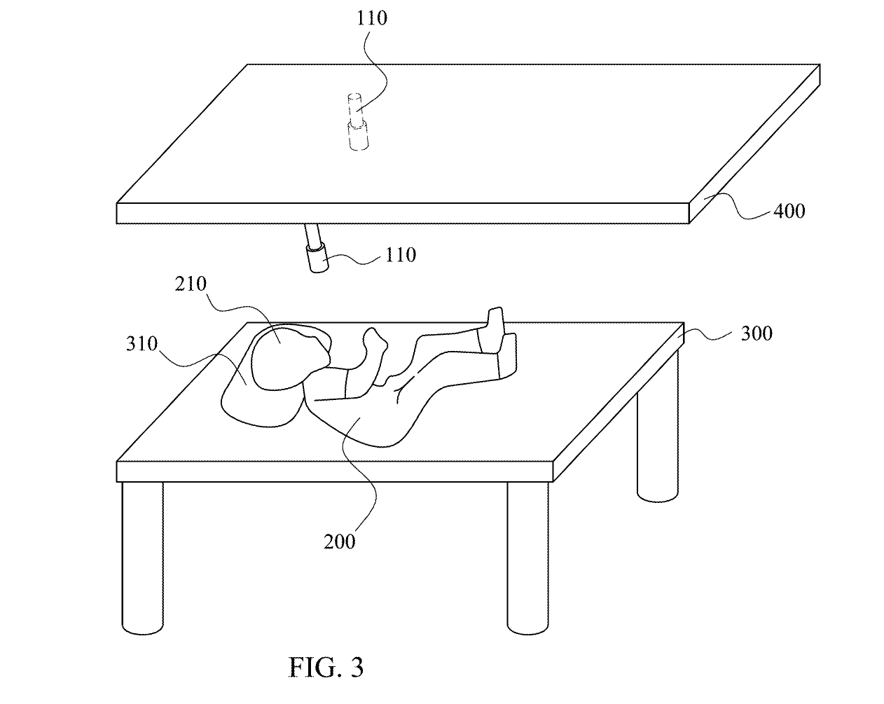

The monitoring system 100 for infant has the features of real-time monitoring and sending warnings, so the monitoring system 100 for infant is very suitable for monitoring infants, together with reference to FIG. 3. FIG. 3 is a schematic diagram of the monitoring system 100 for infant monitoring a monitored person 200, according to one embodiments of the present disclosure, and the monitored person 200 in FIG. 3 is given as an infant for illustration.

As shown in FIG. 3, the monitored person 200 is lying down on a bed 300, and a head 210 of the monitored person 200 is on a pillow 310. The image sensor 110 of the monitoring system 100 for infant is installed on a ceiling 400, right above the monitored person 200 who is lying down, and lens of the image sensor 110 faces toward the monitored person 200. It should be noted that the position where the image sensor 110 is installed is not limited to the above illustration. In other embodiment, the position, angle and the number of the image sensor can be, in accordance with the actual implements, different. In this embodiment, setting the image sensors 110 at two different angles is exemplified. For example, the image sensors 110 set at two different angles can be installed proportionally to the ceiling 400 with 30 degree inwardly, one of the groups being indicated with dash line that was positioned under the ceiling 400, in order to capture the face images of different angles and determine which image among the face images of different angles has more face images.

In one embodiment, the image sensor 110 can be a camera, a camcorder or a video tape recorder, etc. in order to capture the face images.

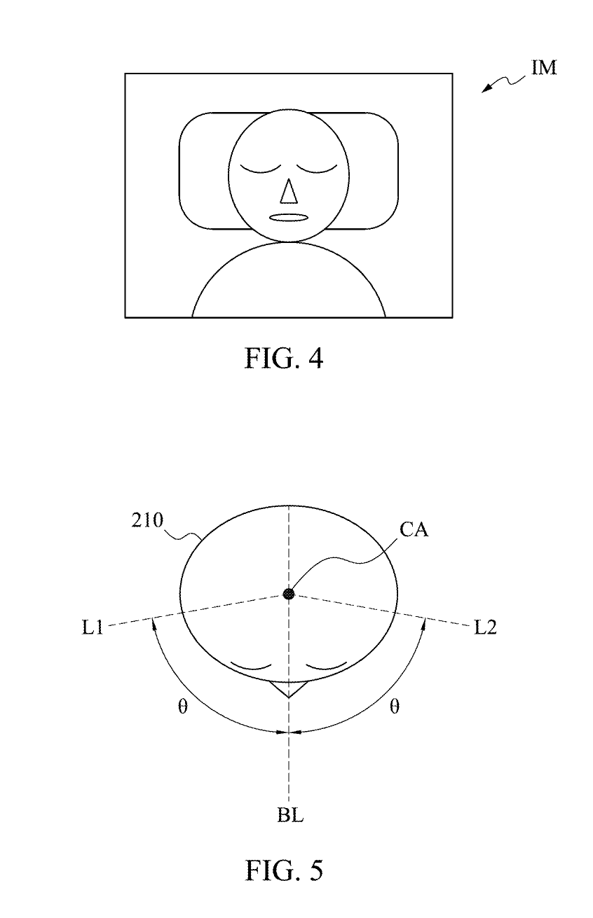

Referring back to the FIG. 1 and FIG. 2, in the step S110, the image sensor 110 can capture a plurality of images IM consecutively. Specifically, with reference to FIG. 4, FIG. 4 is a schematic diagram of the image IM, according to one embodiments of the present disclosure.

In the step S120, the face region determination module 121 determines whether the image IM includes a face region ROF. Specifically, with reference to FIG. 5, FIG. 5 is a schematic diagram of a range of a frame that can be selected for the face region ROF, according to one embodiments of the present disclosure.

Furthermore, the range of a frame that can be selected for the face region ROF is defined as follows.

First, FIG. 5 is described. FIG. 5 is a top view of the head 210 of the monitored person 200 in which the nose tip 220 of the monitored person 200 faces downward.

Afterwards, a baseline BL is defined. The baseline BL extends in the direction toward which the nose tip 220 of the monitored person 200 faces.

Finally, the range of a frame that can be selected for the face region ROF is defined. The range of a frame that can be selected for the face region ROF is provided based on the reference of baseline BL, with 80-degree rotation in clockwise and counterclockwise directions around a center axis CA of the head 210 of the monitored person 200 to a first line L1 and a second line L2 respectively. The region between the first line and the second line is the region of a frame can that can be selected for the face region ROF.

In short, the baseline BL rotating 80 degree clockwise direction to the first line L1 is equivalent to the head 210 of the monitored person 200 pivoting 80 degree from the front to the right. The baseline BL rotating 80 degree counterclockwise direction to the second line L2 is equivalent to the head 210 of the monitored person 200 pivoting 80 degree from the front to the left.

In other word, the range in which the head 210 of the monitored person 200 pivots from the front to the right by 80 degree and to the left by 80 degree is the region of a frame that can be selected for the face region ROF.

It should be noted that the definition of the range of a frame that can be selected for the face region ROF is not limited thereto. For instance, the angle which the head 210 of the monitored person 200 can pivot can be various angles.

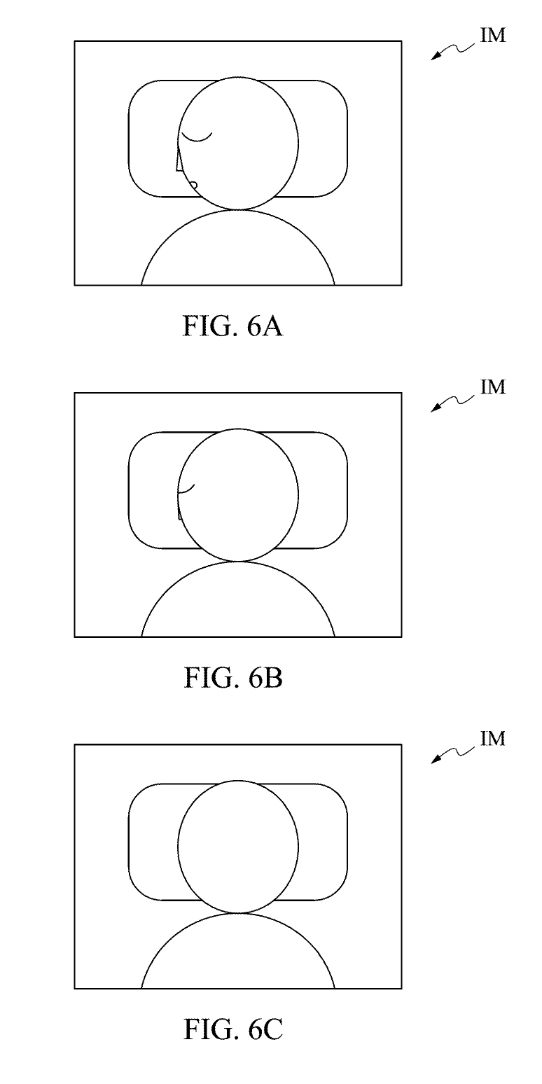

When the face determination module 121 determines that the image IM does not include the face region ROF, the step S130 is performed. Specifically, the reference is now made to FIG. 6A and FIG. 6C. FIG. 6A and FIG. 6C are the schematic diagrams of a plurality of images IM, without the face region ROF, captured by the sensor 110 of the monitoring system 100 for infant, according to one embodiments of the present disclosure.

As shown in FIG. 6A to FIG. 6C, the images IM includes images IM of the monitored person 200 lying prone on the bed and the head 210 of the monitored person 200 pivots to the left as illustrated in FIG. 6A, slightly to the left as illustrated in FIG. 6B and in the prone position as illustrated in FIG. 6C respectively. In the three pivot states as described above, the area that the head 210 of the monitored person 200 pivots is out of the range of a frame that can be selected for the face region ROF. Hence, the face region determination module 121 cannot select a frame for the face region ROF from the image IM. Therefore, it indicates that the image IM does not include the face region ROF.

When the face determination module 121 determines that the image IM does not include the face region ROF, it indicates that the monitored person 200 is in a prone sleeping position or in other sleeping position that may cause the monitored person 200 suffocated. The face determination module 121 will output, in accordance with the step S130, a first warning information WI1 to inform the relevant people to eliminate the condition. For instance, the relevant people adjust the sleeping position of the monitored person 200. The first warning information WI1 may be an audio warning information, a text warning information or a warning information with combination of audio and text.

In one embodiment, outputting the first warning information WI1 includes transmitting the first warning information WI1 to the server 130 as a historical record to inform the relevant people. In other embodiment, outputting the first warning information WI1 includes transmitting the first warning information WI1 via the internet to the relevant people's mobile devices (i.e., cellphone) as a historical record to inform the relevant people. It should be noted that the way of outputting the first warning information WI1 is not limited thereto.

When the face determination module 121 determines that the image IM includes the face region ROF, Process the step S150, the human face identification module 121a can identify the identity of the monitored person in order to extract the past historical information.

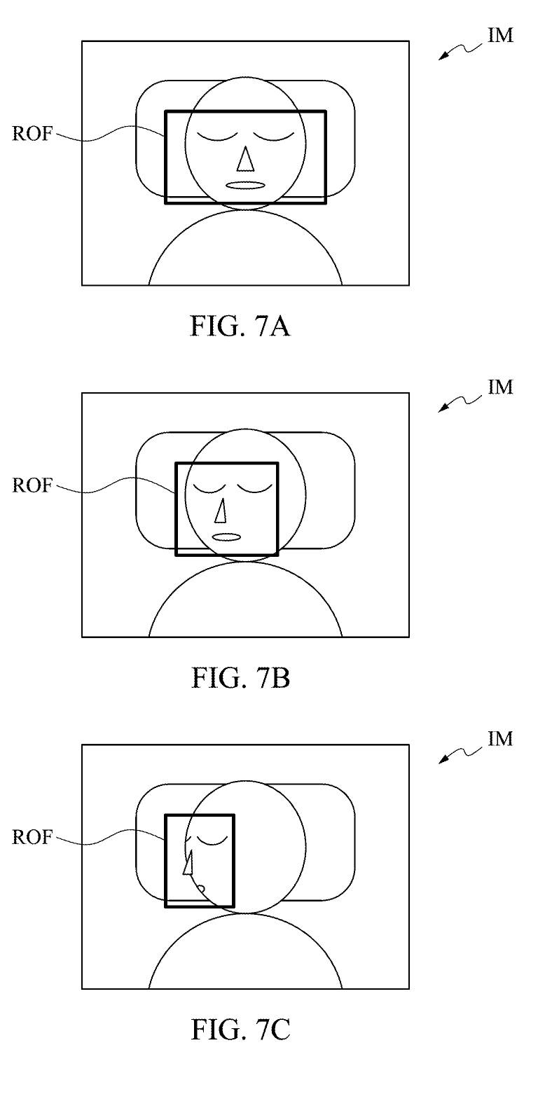

In the step S160, specifically, the reference is now made to FIG. 7A and FIG. 7C. FIG. 7A and FIG. 7C are the schematic diagrams of a plurality of images IM, the face region ROF within, captured by the sensor 110 of the monitoring system 100 for infant, according to one embodiments of the present disclosure.

As shown in FIG. 7A to FIG. 7C, the image IM includes an image IM of the monitored person 200 lying supine on the bed and the head 210 of the monitored person 200 is in supine position as illustrated in FIG. 7A, slightly pivots to the right as illustrated in FIG. 7B and pivots to the right as illustrated in FIG. 7C, respectively. In the three pivot states as described above, the area that the head 210 of the monitored person 200 pivots is within the range of a frame that can be selected for the face region ROF. Hence, the face region determination module 121 can select a frame for the face region ROF from the image IM. Therefore, it indicates that the image IM includes the face region ROF.

When the face determination module 121 determines that the image IM includes the face region ROF, the non-connect physiological information measurement module 122, in accordance of the step S160, calculates a color difference of each pixel of the face region ROF of the images IM captured in sequence to output a physiological information PI, wherein the physiological information PI includes a heart rate, variation of the heart rate and respiration information. The heart rate is the number of times a heart beats per minute. In detail, the non-contact physiological information measurement module 122 calculates the face region ROF captured in sequence to obtain the position of each pixel in the next moment. In detail, the red, green and blue signals of corresponding pixel in the face region ROF in the previous moment are subtracted from those in the next moment, respectively. The corresponding difference values in red, green and blue signals, which are indicated by the difference value of red, green and blue signals are dR, dG and dB respectively can be obtained. Furthermore, by performing addition and subtraction operation of the difference value of red signal dR, the difference value of green signal dG and the difference value of blue signal dB, a horizontal difference value dX and a vertical difference value dY can be obtained. Finally, calculation of the average for each one pixel is performed, and the heartbeat waveform is thus obtained and the physiological information PI can be, by the heartbeat waveform, calculated. The relating method for calculating the color differences can be implemented with other numbers of way, so this disclosure is not limited thereto. In general, while the heart in human body is contracting and relaxing, the blood pressure will be various in capillaries and the face region ROF is a region in which the capillaries are densely distributed, so that the color of the face region ROF may change slightly with the contraction and relaxation of the heart. The non-contact physiological information measurement module 122 provided in the present disclosure utilizes the color difference of the face region ROF to detect the physiological information PI. It should be noticed that the physiological information PI includes heartbeat number but is not limited thereto. In other embodiment, the physiological information can also include the heartbeat number, the resting heart rate, maximum heart rate, the variation of the heartbeat and other number, but this disclosure is not limited thereto.

In one embodiment, outputting the physiological information PI includes transmitting the physiological information PI to the server 130 as a historical record to inform the relevant people. In other embodiment, outputting the physiological information PI includes transmitting the physiological information PI via the internet to the relevant people's mobile devices (i.e., cellphone) as a historical record to inform the relevant people. It should be noted that the way of outputting the physiological information PI is not limited thereto.

After the step S160, the step S170 or the step S180 is performed.

In the step S170, the physiological information determination module 123 determines the heart rate, the variation of the heart rate and a respiration frequency included in the physiological information PI. When the heart rate, the variation of the heart rate and the respiration frequency are not in a reference range, a second warning information WI2 is outputted. As an embodiment, the reference range is the average heart rate range of the monitored person 200 recorded in the past. For instance, a heart beats 120 to 140 times per minute. In other word, when the heart rate included in the physiological information PI is not in the range of 120 to 140 times per minute, it indicates that condition of the heart rate of the monitored person 200 is abnormal. The physiological information determination module 123 outputs the second warning information WI2 to inform the relevant people to eliminate the condition. For instance, the relevant people diagnose or do some treatment to the monitored person 200. The second warning information WI2 may be an audio warning information, a text warning information or a warning information with combination of audio and text.

In one embodiment, the variation of the heart rate included in the physiological information PI is obtained by the heartbeat waveform measured from the color difference by the non-contact physiological information measurement module 122 calculating a time series relationship between the adjacent heartbeats. The variation of the heart rate can be used to analyze the status of the balance of autonomic nerve, which is effective to detect the unknown reason of the cardiogenic sudden death of infants.

In one embodiment, the physiological information PI includes the respiration information. The non-contact physiological information measurement module 122 can perform a Fourier Transformation with the heartbeat waveform and then conduct frequency-domain analysis to obtain the respiration information. The maximum peak value in the frequency spectrum is a heartbeat signal and the second peak value is a respiration signal.

In one embodiment, outputting the second warning information WI2 includes transmitting the second warning information WI2 to the server 130 as a historical record to inform the relevant people. In other embodiment, outputting second warning information WI2 includes transmitting the second warning information WI2 via the internet to the relevant people's mobile devices (i.e., cellphone) as a historical record to inform the relevant people. It should be noted that the way to output the second warning information WI2 is not limited thereto.

In the step S180, the physiological information determination module 123 determines the heart rate included in the physiological information PI. When a difference value of the heart rates included in the physiological information PI outputted in sequence exceeds a threshold value, the physiological information determination module 123 outputs a third warning information WI3. Specifically, the difference value of the heart rates indicates the difference value between the heart rate of the monitored person 200 measured one second ahead and one second later. When the difference value is too large, it indicates that the heart rate of the monitored person 200 is not stable. For example, the heart rate of the monitored person 200 measured one second ahead is 120 times per minute and one second later is 130 times per minute, then the difference value of the heart rate of the monitored person 200 is 10. When the threshold value is set to 5, the difference value exceeds the threshold value, indicating that the condition of the heart rate of the monitored person 200 is abnormal. The physiological information determination module 123 outputs a third warning information WI3 to inform the relevant people to eliminate the condition. For instance, the relevant people diagnose or do some treatment to the monitored person 200. The third warning information WI3 may be an audio warning information, a text warning information or a warning information with combination of audio and text.

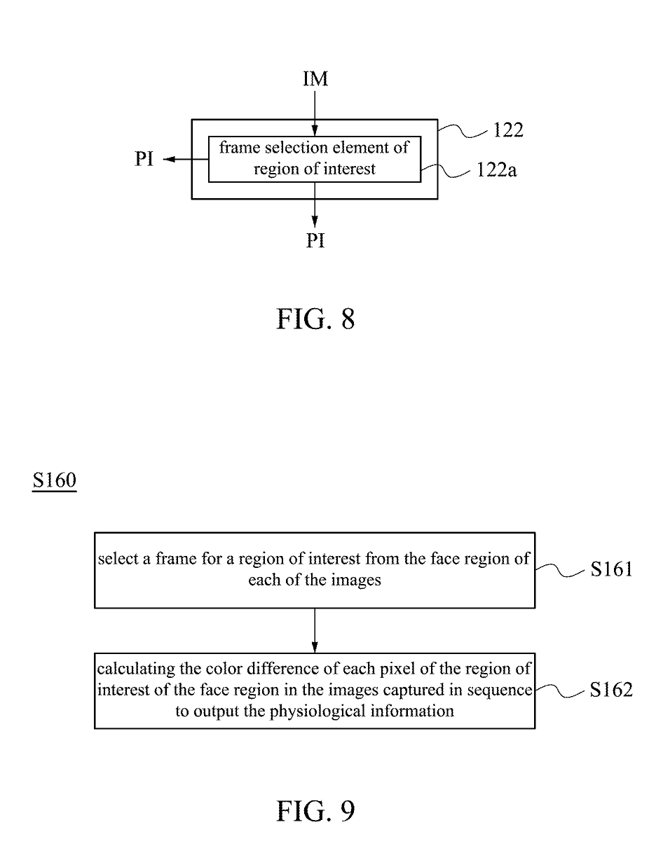

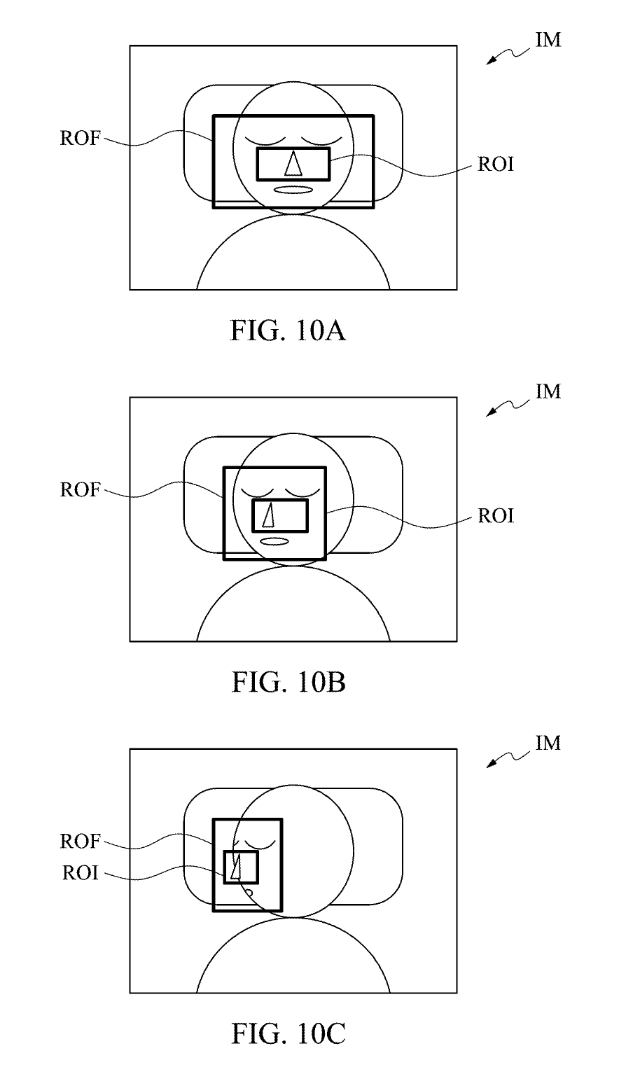

Reference is made back to FIG. 8, FIG. 9 and FIG. 10A to FIG. 10C. FIG. 8 is a further function block diagram of the non-contact physiological information measurement module 122 of the monitoring system 100 for infant, according to one embodiments of the present disclosure. FIG. 9 is a further flowchart of the step S160 of the monitoring method for infant 100, according to one embodiments of the present disclosure. FIG. 10A to FIG. 10C are the schematic diagrams of a region of interest ROI according to one embodiments of the present disclosure.

As shown in FIG. 8, the non-contact physiological information measurement module 122 further includes a frame selection element of the region of interest 122a. As shown in FIG. 9, the step S160 further includes the step S161 and S162.

The frame selection element of the region of interest 122a selects a frame, in accordance with the step S161, for the region of interest ROI from the face region ROF of the image IM and calculates, in accordance with the step S162, a color difference of each pixel of the region of interest ROI of the face region ROF in the images IM captured in sequence to output the physiological information PI.

Specifically, in the step S161, the region of interest ROI generally is the region between the eyes and the mouth in the face region ROF. That is, after coordinates of eye feature points and mouth feature point are defined, the frame selection element of the region of interest 122a may select a frame, in accordance of the coordinates of eye feature points and the mouth feature point, for the region of interest ROI from the face region ROF. In addition, since the coordinates of eye feature points and mouth feature point are not the technical features of present disclosure, it will not be described.

In the step S162, the frame selection element of the region of interest 122a can perform the calculation with the regions of interest ROI captured in sequence in order to obtain the physiological information PI and output it. Because the way of obtaining the physiological information PI with the region of interest ROI is the similar to the way of obtaining the physiological information PI with the face region ROF, it will not be described.

Through the way of the frame selection element of the region of interest 122a selecting a frame for the region of interest ROI from the face region ROF of the image IM, the detected area is reduced from a larger range of the face region ROF to a smaller range of the region of interest ROI, so that the operating speed increases.

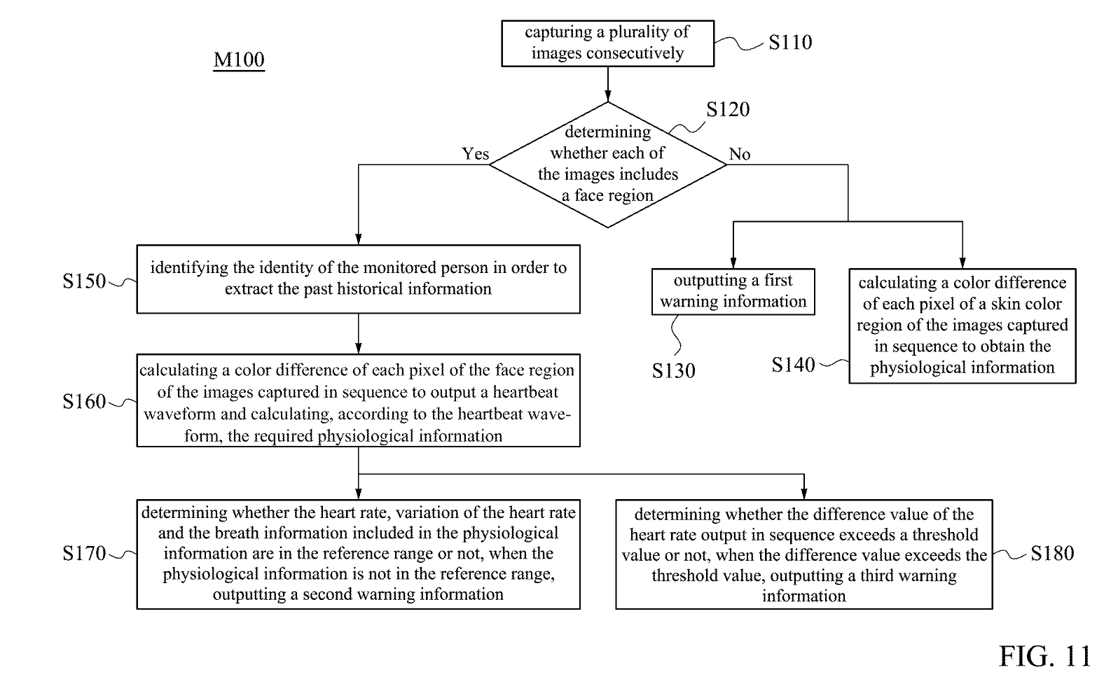

Reference is made to FIG. 1 and FIG. 11. FIG. 11 is a flowchart of a monitoring method for infant M200, according to another embodiment of the present disclosure.

In the present disclosure, the monitoring system 100 for infant may execute the monitoring method M200 for infant to perform monitoring, wherein the monitoring method M200 for infant is almost the same as the monitoring method M100 for infant. The difference is that the monitoring method M200 for infant further includes the step S140. In order to manifest the difference, the resemblance between the monitoring method M200 for infant and the monitoring method M100 for infant will not be described.

When the face region determination module 121 determines that the image IM does not include the face region ROF, the step S140 is performed.

In the step S140, the non-contact physiological information measurement module 122 calculates a color difference of each pixel of a skin color region in the images IM captured in sequence to output the physiological information PI and performs, with the skin color regions captured in sequence, the calculation in order to obtain the physiological information PI and output it. The calculation method can be implemented in many various ways. Because the way of obtaining the physiological information PI with the skin color region is almost the same as the way of obtaining the physiological information PI with the face region ROF, it will not be described.

When the physiological information PI is obtained and outputted, the step S170 and S180 are performed. Because the step S170 and S180 have been illustrated, it will not be described.

It should be noted that the face region determination module 121, the non-contact physiological information measurement module 122 and the physiological information determination module 123 of the monitoring system 100 for infant may be implemented with the hardware, software, firmware or the combination of those.

As the description above, the monitoring system for infant in the present disclosure with the image sensor, the face region determination module, the non-contact physiological information measurement module and the physiological information determination module performs the monitoring method for infant including the steps from S110 to S180. The monitoring method for infant can be configured to assist the nurses to monitor the infants' health condition, when the infants' health conditions are not stable, outputting the warning information to avoid the accidents. The monitoring system for infant may be connected to the server to filing for the health care workers' and the parents' inquiries in convenience. The monitoring system for infant with remote image sensor (i.e., video recorder) can monitor the heart rate instantly, and it prevents causing injury induced by wearable devices on the infants' skin. In addition, the image sensor performs the passive measurement and does not generate the extra energy of electromagnetic wave which may cause influence on the infants.

It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims.

What is claimed is:

1. A monitoring system for infant, comprising:
one or more image sensors configured to capture a plurality of images consecutively;
a face region determination module configured to determine whether each of the images includes a face region, wherein when each of the images does not include the face region, the face region determination module outputs a first warning information;
a human face identification module, when each of the images includes the face region, configured to identify the identity of a monitored person in the images in order to extract the past historical information corresponding to the identity of the monitored person;
a non-contact physiological information measurement module, when each of the images includes the face region, configured to calculate a color difference of each pixel of the face region of the images captured in sequence to output a heartbeat waveform and calculate, according to the heartbeat waveform, a required physiological information; and
a physiological information determination module configured to determine a heart rate, variation of the heart rate, respiration information included in the physiological information, wherein when the physiological information is not in a reference range, the physiological information determination module outputs a second warning information.

2. The monitoring system for infant of claim 1, wherein the non-contact physiological information measurement module comprises a frame selection element of region of interest configured to select a frame for a region of interest from the face region of each of the images, when the face region determination module determines that each of the images includes the face region, the non-contact physiological information measurement module being configured to calculate a color difference of each pixel of the region of interest in the face region of the images captured in sequence to output the physiological information.

3. The monitoring system for infant of claim 1, wherein when a difference value of the heart rates included in the physiological information outputted in sequence exceeds a threshold value, the physiological information determination module outputs a third warning information.

4. The monitoring system for infant of claim 1, wherein when each of the images does not include the face region, the non-contact physiological information measurement module is configured to calculate a color difference of each pixel of a skin color region of the images captured in sequence to output the physiological information.

5. The monitoring system for infant of claim 1, wherein the one or more image sensors are configured to capture the face images of the monitored person including the monitored person.

6. The monitoring system for infant of claim 5, wherein one of the image sensors is set at a first angle and another one of the image sensors is set at a second angle, in order to obtain the images of a plurality of angles, and wherein the face region determination module determines the images including more information of human face among the images of the angles.

7. The monitoring system for infant of claim 1, wherein the non-contact physiological information measurement module calculates, by the heartbeat waveform, a time series relationship between the adjacent heartbeats in order to obtain the variation of the heart rate.

8. The monitoring system for infant of claim 1, wherein the non-contact physiological information measurement module performs a calculation with the heartbeat waveform in order to obtain a frequency-domain analysis corresponding to the heartbeat waveform, and further obtain the respiration information.

9. A monitoring method for infant, comprising:
capturing a plurality of images consecutively;
determining whether each of the images includes a face region;
when each of the images does not include the face region, outputting a first warning information;
when each of the images includes the face region, identifying the identity of a monitored person in order to extract the past historical information;
when each of the images includes the face region, calculating a color difference of each pixel of the face region of the images captured in sequence to output a heartbeat waveform, and calculating, by the heartbeat waveform, a required physiological information; and
determining whether a heart rate, variation of the heart rate and respiration information included in the physiological information are in a reference range, when the physiological information is not in the reference range, outputting a second warning information.

10. The monitoring method for infant of claim 9, wherein when determining each of the images includes the face region, the procedure of calculating, according to the face region of each of the images, the physiological information comprises:
selecting a frame for a region of interest from the face region of each of the images, and
calculating a color difference of each pixel of the region of interest in the face region of the images captured in sequence to output the physiological information.

11. The monitoring method for infant of claim 9, further comprising: determining whether a difference value of the heart rates included in the physiological information outputted in sequence exceeds a threshold value, when the difference value exceeds the threshold value, outputting a third warning information.

12. The monitoring method for infant of claim 9, wherein when each of the images does not include the face region, calculating a color difference of each pixel of a skin color region of the images captured in sequence to obtain the physiological information.

13. The monitoring method for infant of claim 9, wherein outputting the physiological information comprises transmitting the physiological information to a server, outputting the first warning information comprises transmitting the first warning information to the server and outputting the second warning information comprises transmitting the second warning information to the server.

14. The monitoring method for infant of claim 9, wherein capturing a plurality of images consecutively comprises capturing, for the monitored person, the face images of the monitored person.

15. The monitoring method for infant of claim 14, wherein capturing a plurality of images consecutively comprises obtaining the images of a plurality of angles and determining the images including more information of human face among the images of the angles.

16. The monitoring method for infant of claim 9, wherein calculating, by the heartbeat waveform, the required physiological information comprises calculating, by the heartbeat waveform, a time series relationship between the adjacent heartbeats in order to obtain the variation of the heart rate.

17. The monitoring method for infant of claim 9, wherein calculating, by the heartbeat waveform, the required physiological information comprises performing a calculation with the heartbeat waveform in order to obtain a frequency-domain analysis corresponding to the heartbeat waveform, and further obtain the respiration information.
